(19) United States
(12) Patent Application Publication
(10) Pub. No.: US 2022/0032091 A1
(43) Pub. Date: Feb. 3, 2022

(54) FILTER MASK WITH ACCESSORY BELT

(71) Applicant: GIFTEDNESS AND CREATIVITY COMPANY, Safat (KW)

(72) Inventor: HASSAN A A A AL-JAFAR, SAFAT (KW)

(21) Appl. No.: 17/352,226

(22) Filed: Jun. 18, 2021

Related U.S. Application Data
(60) Provisional application No. 63/057,906, filed on Jul. 29, 2020.

(51) Int. Cl. A62B 7/10; A61F 9/02; A61L 2/26; A62B 18/08; A62B 18/00; A62B 18/02; A62B 23/02; A62B 9/00; A41D 13/11

(52) U.S. Cl. CPC A62B 7/10; A61F 9/02; A61L 2/26; A62B 18/084; A62B 18/006; A61L 2202/16; A62B 18/025; A62B 23/02; A62B 9/00; A41D 13/11; A62B 18/08

(57) ABSTRACT

The filter mask with accessory belt combines a filter mask with powered exhaust fans and an accessory belt for carrying protective and/or medical equipment, as well as a power source for the exhaust fans. The filter mask includes a rigid mask body having opposed outer and inner faces and at least one exhaust vent formed therein. An inhalation port is formed through the mask body, and a filter covers the inhalation port. At least one exhaust fan is mounted on the inner face of the mask body adjacent the at least one exhaust vent. At least one head securement strap is attached to the mask body. The accessory belt includes at least one pocket. A power supply is received within the at least one pocket of the accessory belt. The power supply is in electrical communication with the at least one exhaust fan for providing power thereto.

### CROSS-REFERENCE TO RELATED APPLICATION

This application claims the benefit of U.S. Provisional Patent Application No. 63/057,906, filed on Jul. 29, 2020.

### BACKGROUND

### 1. Field

The disclosure of the present patent application relates to personal protective equipment, and particularly to a filter mask with a wearable accessory belt that has with a powered fan in the mask, the accessory belt being adapted for carrying a power supply for the powered fan and additional protective equipment.

### 2. Description of the Related Art

Disposable face masks and respirators are well known in the art. In the medical field, such masks are used in preventing contamination of a patient from the exhaled breath of healthcare personnel. In recent years, with increased concern for infection of healthcare personnel by airborne pathogens, such masks have become important in preventing infection of healthcare personnel as well. Healthcare personnel now increasingly show a preference for wearing face masks that form a barrier or seal between the periphery of the mask and the face of the wearer. As a result of this sealing engagement between the mask and wearer's skin, substantially all air inhaled or exhaled by the wearer is forced to flow through the filter media of the face mask. As a result, such filter masks can become hot and uncomfortable to the wearer during periods of long-term wear, and breathability can also be a problem.

In addition to the above, since all airflow is generated by the wearer of the mask, there is an alternation of environmental air flowing inward through the mask, and exhaled air flowing out through the same filter material of the mask. Since no filter is perfect, some small portion of airborne contaminants may still pass through the filter material of the mask, drawn in by the wearer's inhalation. Additionally, unwanted materials from the wearer, such as odor-causing bacteria, mucus and the like, can accumulate on the inner surface of the filter material, since the wearer is directly breathing on this material.

The above properties of conventional filter masks are compounded by the fact that masks are only one form of personal protective equipment and must be used in conjunction with a variety of other equipment, such as gloves, sanitizers and the like. Each of these additional items must be gathered and separately carried. Thus, a filter mask with accessory belt solving the aforementioned problems is desired.

### SUMMARY

The filter mask with accessory belt combines a filter mask with powered exhaust fans and an accessory belt for carrying protective and/or medical equipment, as well as a power source for the exhaust fans. The filter mask includes a rigid mask body adapted for covering a mouth and nose portion of the user's face. A peripheral seal may be secured to a periphery of the mask body to form an airtight seal against the user's face. The mask body has opposed outer and inner faces, and at least one exhaust vent is formed through the mask body. An inhalation port is further formed through the mask body. The inhalation port may be formed through a lower end of the mask body. A filter covers the inhalation port for filtering air flowing into the mask body through the inhalation port for breathing by the user.

At least one exhaust fan is mounted on the inner face of the mask body adjacent the at least one exhaust vent. The at least one exhaust fan blows the user's exhaled air from within the mask body to the external environment. At least one strap is attached to the mask body for holding the filter mask on the user's head. Additionally, a filter housing may be provided for covering the inhalation port and receiving the filter.

The accessory belt may be any suitable type of belt adapted to be worn by the user. The accessory belt may be adapted to be worn about the user's waist. The accessory belt includes a belt portion and at least one pocket attached to the belt portion. A power supply is disposed within the at least one pocket of the accessory belt. The power supply is in electrical communication with the at least one exhaust fan for providing power thereto. It should be understood that as many pockets as desired may be attached to the accessory belt, and that the pockets may be used to carry any desired personal protective and/or medical equipment. Examples of items that may be carried in the pockets include a thermometer, a sanitizer dispenser, protective gloves, a bottle of water, and the like. It should be understood that any additional or alternative items may be carried, such as a bar code reader for reading patient data, tissues or drying papers, an electronic device for gathering temperature and other patient data, etc.

Additionally, protective goggles or glasses may be provided to cover the user's eyes. For further protection, an auxiliary respirator mask may be provided for releasably covering the filter mask. In addition to providing an added layer of filtration, the auxiliary respirator mask covers the exhaust vents, thus creating a region of positive pressure between the mask body and the auxiliary respirator mask when the exhaust fans are blowing. This positive pressure region (i.e., a region in which the air pressure is greater than that of the ambient environment) prevents viruses, bacteria, etc. from traveling inward through the auxiliary respirator mask and the exhaust vents. Additionally, the outwardly blown air acts to blow airborne contaminants, virus-containing droplets and the like away from the user's face.

These and other features of the present subject matter will become readily apparent upon further review of the following specification.

### BRIEF DESCRIPTION OF THE DRAWINGS

FIG. 1 is an environmental perspective view of a filter mask with accessory belt.

FIG. 2 is an environmental perspective view of the filter mask of the filter mask with accessory belt.

FIG. 3 is a partially-exploded perspective view of the filter mask of FIG. 2 as seen from behind the mask.

FIG. 4 is a partially-exploded perspective view of the filter mask of FIG. 2, showing components of the air intake port.

FIG. 5 is a block diagram of the power and control electronics of the filter mask with accessory belt.

Similar reference characters denote corresponding features consistently throughout the attached drawings.

### DETAILED DESCRIPTION OF THE PREFERRED EMBODIMENTS

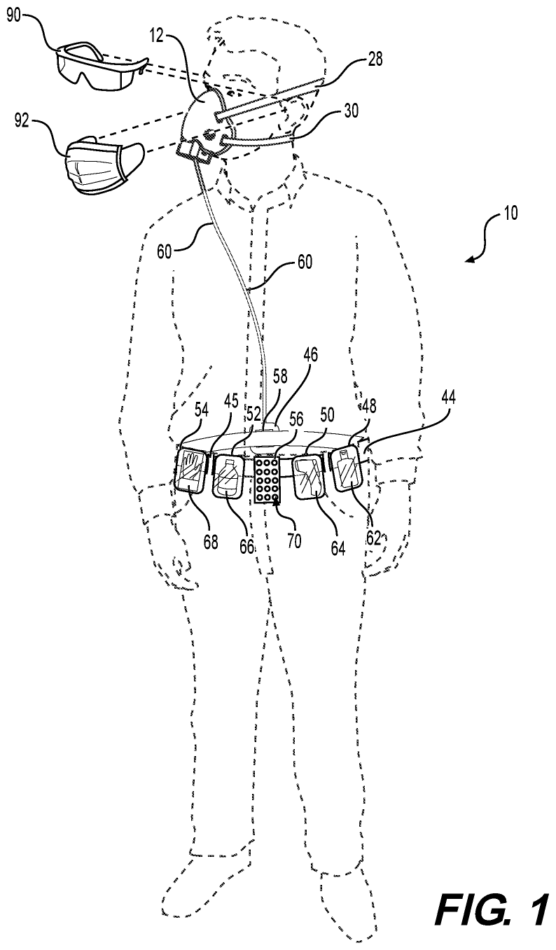

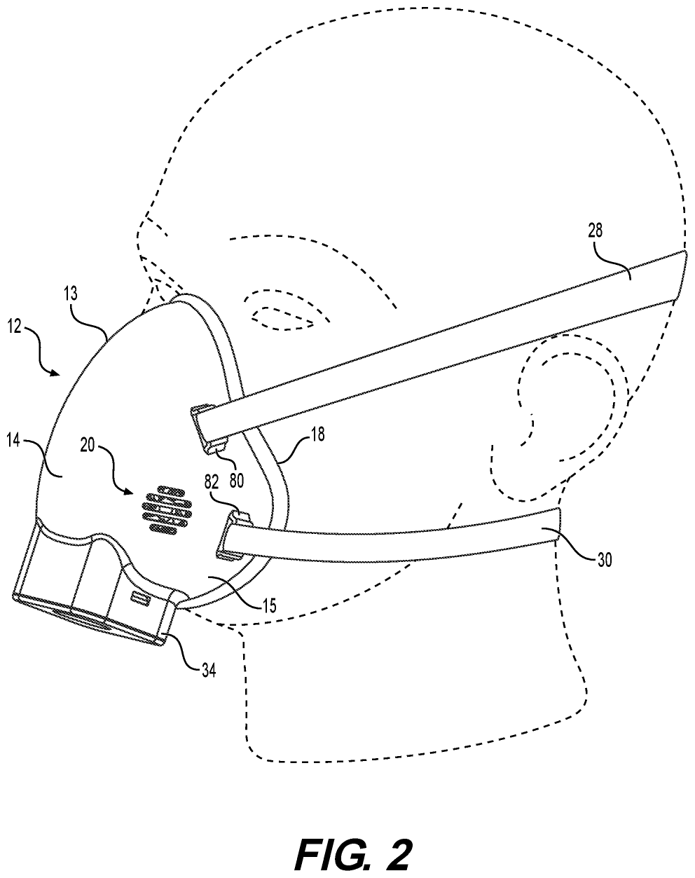

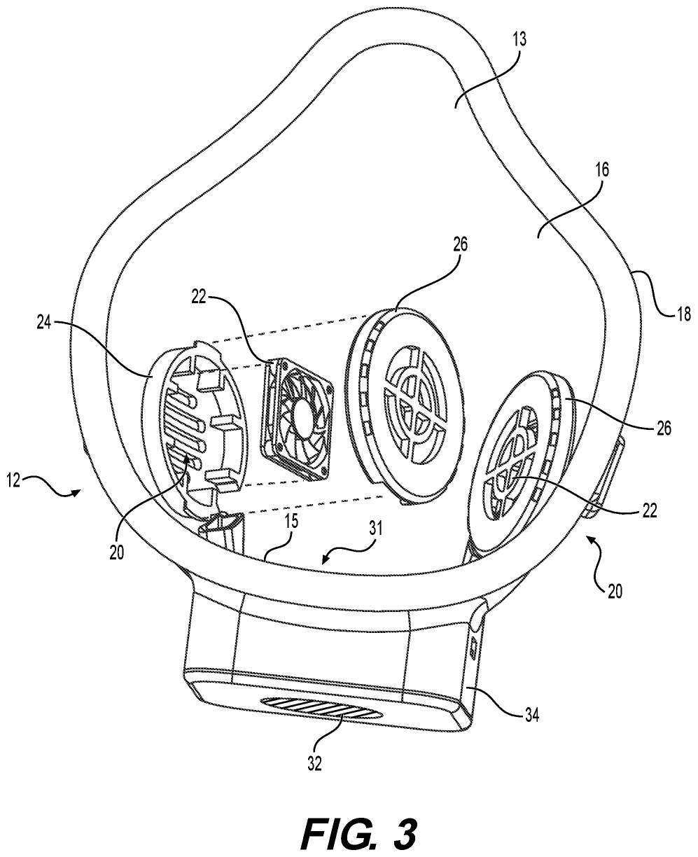

As shown in FIG. 1, the filter mask with accessory belt, designated generally as 10 in the drawings, combines a filter mask 12 with powered exhaust fans and an accessory belt 44 for carrying protective and/or medical equipment, as well as a power source 58 for the exhaust fans. As best seen in FIGS. 2 and 3, the filter mask 12 includes a rigid mask body 13 made of plastic dimensioned and configured for covering a mouth and nose portion of the user's face. A peripheral seal 18 may extend around the periphery of the mask body 13 to form an airtight seal against the user's face. The mask body 13 has opposed outer and inner faces 14, 16, respectively, and at least one exhaust vent 20 is formed through the mask body 13. In the exemplary mask 13 of FIG. 3, two such vents 20 are provided, although it should be understood that any suitable number of exhaust vents may be formed in the mask body 13. It should be further understood that the positioning, overall shape and relative dimensions of the exhaust vents 20 are shown for exemplary purposes only. Additionally, it should be understood that the shape, overall configuration and relative dimensions of the mask body 13 are shown for exemplary purposes only, provided that the mask body 13 covers the user's nose and mouth.

Additionally, an inhalation port 31 is formed in the mask body 12. The inhalation port 31 may be formed through a lower end 15 of the mask body 13. As will be described in greater detail below, a filter 32 covers the inhalation port 31 for filtering air flowing into the mask body 13 through the inhalation port 31 for breathing by the user.

At least one exhaust fan 22 is mounted on the inner face 16 of the mask body 13 adjacent the at least one exhaust vent 20. In the exemplary mask body 13 of FIG. 3, two such exhaust fans 22 are provided. In FIG. 3, the exhaust fans 22 are each mounted within a corresponding fan housing 24 and covered by a removable vented cover 26. However, it should be understood that the fan housings 24 and removable vented covers 26 are shown for exemplary purposes only, and that exhaust fans 22 may be mounted to the mask body 13 by any suitable type of mounting or attachment. Additionally, it should be understood that the exhaust fans 22 may be any suitable type of fans, such as conventional small fans powered by a universal serial bus (USB) connection or the like, as are commonly used by hobbyists, used in personal electronic devices, etc.

The at least one exhaust fan 22 blows the user's exhaled air from within the mask body 13 to the external environment through the at least one exhaust vent 20. As shown in FIG. 2, at least one strap is attached to the mask body 13 for holding the filter mask 12 on the user's head. In FIG. 2, two such straps 28, 30 are shown. However, it should be understood that the pair of straps 28, 30 are shown for exemplary purposes only, and that any suitable number of straps may be used. Further, in FIG. 2, the straps 28, 30 are shown as wrapping around the user's head. It should be understood that any suitable type of straps, such as ear straps or the like, may be used. Additionally, it should be understood that the straps 28, 30 may be attached to the mask body 13 by any suitable type of attachment or mounting, and the retainers 80, 82 shown in FIG. 2 are shown for exemplary purposes only.

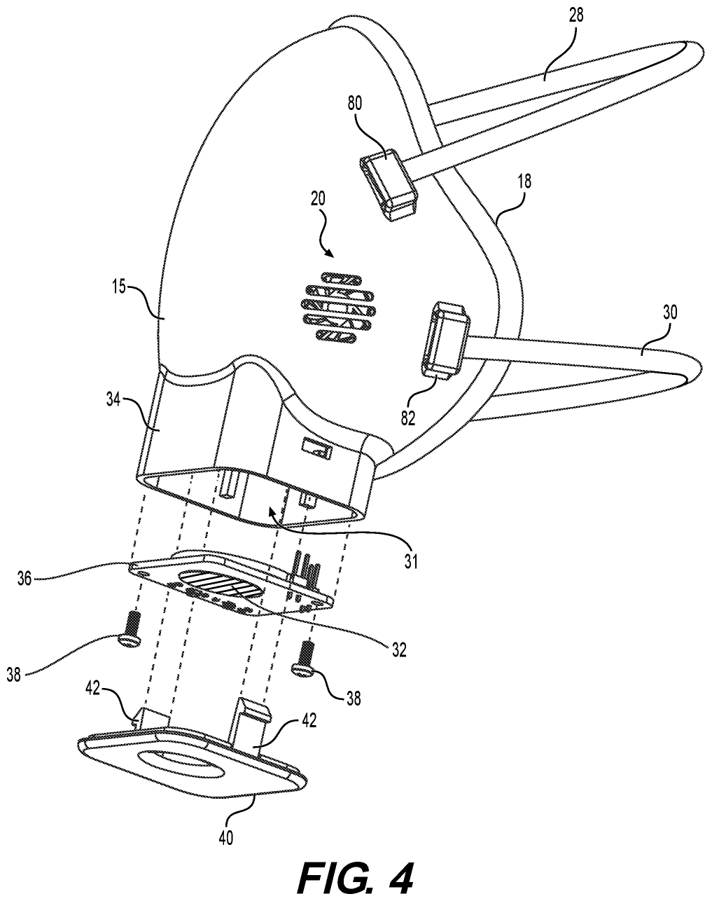

As best seen in FIG. 4, a filter housing 34 may be provided for covering the inhalation port 31 and receiving the filter 32. In order to make filter 32 easy to remove for replacement and/or washing thereof, the filter 32 may be mounted on a removable plate 36, as shown, the plate 36 being removably mounted within the filter housing 34. The removable plate 36 may be secured within the filter housing 34 by screws 38 or other fasteners. It should be understood that the overall configuration and relative dimensions of both the filter housing 34 and the filter plate 36 are shown for exemplary purposes only. Additionally, it should be understood that the screws 38 are shown for exemplary purposes only, and that removable plate 36 may be secured to the filter housing 34 by any suitable type of fasteners. Further, a removable cover 40 may be used to provide additional protection and stability for the filter 32 within the filter housing 34. In FIG. 4, the removable cover 40 is shown as including clips 42 for releasably securing removable cover 40 to the filter housing 34, although it should be understood that the clips 42 are shown for exemplary purposes only, and that any suitable method of releasable attachment may be used to removably secure the removable cover 40 to the filter housing 34. It should be understood that the filter 32 may be any suitable type of filter for filtering out dust, allergens, bacteria, viruses, and the like.

Returning to FIG. 1, the accessory belt 44 may be any suitable type of belt adapted to be worn by the user. For example, the accessory belt 44 may be adapted to be worn about the user's waist. The accessory belt 44 includes a belt portion 45 and at least one pocket attached to the belt portion 45. In FIG. 1, five such pockets 46, 48, 50, 52 and 54 are shown, although it should be understood that any desired number of pockets may be attached to the belt portion 45. Additionally, it should be understood that the style, shape, and relative dimensions of belt portion 45 and pockets 46, 48, 50, 52 and 54 are shown in FIG. 1 for exemplary purposes only. It should be understood that the belt portion 45 and pockets 46, 48, 50, 52, 54 may be made of any suitable material, for example, a plastic material that may be easily cleaned and is microwave-safe for microwave sterilization.

In FIG. 1, a power supply 58 is stored within the rear pocket 46. The power supply 58 is in electrical communication with the exhaust fans 22 via cables 60 for providing power thereto. It should be understood that the power supply 58 may be any suitable type of power supply, such as a rechargeable battery with a solar charger or the like. The additional pockets 48, 50, 52, and 54 are provided for carrying personal protective and/or medical equipment. It should be understood that any desired items may be carried in pockets 48, 50, 52 and 54. In FIG. 1, pocket 50 carries a non-contact thermometer 64, pocket 48 carries a sanitizer dispenser 62 (which may be a conventional sanitizer dispenser or a powered sanitizer dispenser), pocket 54 carries protective gloves 68 (such as conventional disposable nitril gloves or the like), and pocket 52 carries a bottle of water 66. It should be understood that any additional or alternative items may be carried, for example, a bar code reader for reading patient data, tissues or drying papers, an electronic device for gathering temperature and other patient data, etc. It should be further understood that electronic items carried within the pockets 48, 50, 52, 54 may be charged using the power supply 58. For example, the thermometer 64 may be an electronic thermometer and may be recharged through connection with the power supply 58. In one pocket, a finger pulse oximeter could be added. The pulse oximeter is a vital sign device to check the blood oxygen saturation in patients with COVID-19 chest problems.

Additionally, as shown, protective goggles 90 or glasses may be provided to cover the user's eyes. It should be understood that protective goggles 90 are shown in FIG. 1 for exemplary purposes only, and that any suitable type of eye protection may be used. For further protection, an auxiliary respirator mask 92, such as a conventional N95 mask, a surgical mask or the like, may be provided for releasably covering the filter mask 12. In addition to providing an added layer of filtration, the auxiliary respirator mask covers the exhaust vents 20, thus creating a region of positive pressure between the mask body 13 and the auxiliary respirator mask 92 when the exhaust fans 22 are blowing. This positive pressure region (i.e., a region in which the air pressure is greater than that of the ambient environment) prevents viruses, bacteria, etc. from traveling inward through the auxiliary respirator mask 92 and the exhaust vents 20. Additionally, the outwardly blown air acts to blow airborne contaminants, virus-containing droplets and the like away from the user's face.

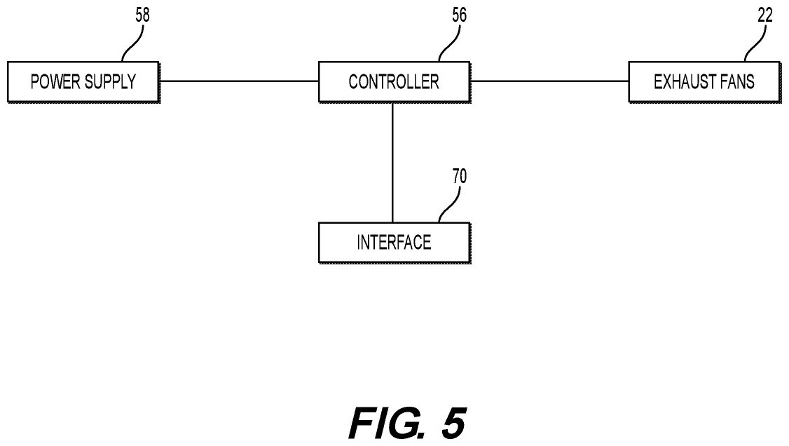

As shown in FIGS. 1 and 5, a controller 56 may be attached to the accessory belt 44 for controlling the operation of the exhaust fans 22. It should be understood that the interface 70 shown in FIG. 1 is shown for exemplary purposes only, and may be any suitable type of control interface for use by the user, such as push buttons, switches, a touchscreen, or the like. It should be further understood that the controller 56 may be any suitable type of controller, such as control circuitry, a processor or the like, allowing the user to selectively control operation of the exhaust fans 22.

It is to be understood that the filter mask with accessory belt is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A filter mask with accessory belt, comprising:
a filter mask including:
a rigid mask body having an outer face, an inner face, at least one exhaust vent defined in the mask body, and an inhalation port extending through the mask body, the mask body being dimensioned and configured for covering a user's mouth and nose;
at least one exhaust fan mounted on the inner face of the mask body adjacent the at least one exhaust vent;
at least one strap attached to the mask body for holding the filter mask on a face of a user, and
a filter covering the inhalation port:
an accessory belt adapted to be worn by the user, the accessory belt including a belt portion and at least one pocket attached to the belt portion; and
a power supply removably disposed within the at least one pocket of the accessory belt, the power supply being in electrical communication with the at least one exhaust fan.

2. The filter mask with accessory belt as recited in claim 1, wherein the at least one pocket comprises a plurality of pockets.

3. The filter mask with accessory belt as recited in claim 2, further comprising a thermometer disposed within a corresponding one of the plurality of pockets.

4. The filter mask with accessory belt as recited in claim 2, further comprising a sanitizer dispenser disposed within a corresponding one of the plurality of pockets.

5. The filter mask with accessory belt as recited in claim 2, further comprising at least one protective glove disposed within a corresponding one of the plurality of pockets.

6. The filter mask with accessory belt as recited in claim 2, further comprising a bottle of water received disposed a corresponding one of the plurality of pockets.

7. The filter mask with accessory belt as recited in claim 1, further comprising protective goggles adapted to be worn on the face of the user.

7. The filter mask with accessory belt as recited in claim 1, further comprising an auxiliary respirator mask releasably covering the filter mask.

8. The filter mask with accessory belt as recited in claim 1, wherein the filter mask further comprises a filter housing covering the inhalation port, the filter housing receiving the filter.

9. The filter mask with accessory belt as recited in claim 1, wherein the inhalation port is formed in a lower end of the mask body.

10. The filter mask with accessory belt as recited in claim 1, wherein the filter mask further comprises a peripheral seal secured to a periphery of the mask body.
